(19) United States
(12) Patent Application Publication
(10) Pub. No.: US 2007/0249960 A1
(43) Pub. Date: Oct. 25, 2007

(54) Biopsy punch

(75) Inventor: Warren P. Williamson IV, Loveland, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/788,209

(22) Filed: Apr. 19, 2007

Related U.S. Application Data
(60) Provisional application No. 60/793,780, filed on Apr. 21, 2006.

(51) Int. Cl. A61B 10/00

(52) U.S. Cl. 600/564

(57) ABSTRACT

A biopsy punch for removing a biopsy sample from a biopsy area of a patient includes a handle and a tubular blade. The handle is adapted for gripping by a user. The tubular blade extends around a longitudinal axis and has first and second longitudinally spaced blade ends separated by a blade body. The tubular blade is operatively coupled to the handle, has a cutting edge at the second blade end, has a plurality of openings located in the blade body and allowing the user to view the biopsy area for positioning the cutting edge in the biopsy area, and has at least one retainer prong extending toward the longitudinal axis and operative to engage the severed sample to assist in retaining the severed sample in the blade body. The tubular blade is adapted to sever at least a portion of the biopsy sample from the biopsy area and retain the severed sample in the blade body. A method of using the biopsy punch is also described.

### RELATED PATENT APPLICATION

This application claims priority to the filing date of U.S. Provisional Application No. 60/793,780, filed Apr. 21, 2006, the subject matter of which is incorporated herein by reference.

### TECHNICAL FIELD

The present invention relates to an apparatus and method for use of a biopsy punch and, more particularly, to a biopsy punch capturing a biopsy sample.

### BACKGROUND OF THE INVENTION

Skin biopsy is one of the most important diagnostic tests for skin disorders. Punch biopsy, considered the primary technique for obtaining diagnostic full-thickness skin specimens, requires basic general surgical and suture-tying skills and is easy to learn. The technique involves the use of a circular blade that is rotated down through the epidermis and dermis, and into the subcutaneous fat, yielding a 3- to 4-mm cylindrical core of tissue sample. Stretching the skin perpendicular to the lines of least skin tension before incision results in an elliptical-shaped wound, allowing for easier closure by a single suture.

Once the specimen is obtained, caution must be used in handling it to avoid crush artifact. Punch biopsies are useful in the work-up of cutaneous neoplasms, pigmented lesions, inflammatory lesions and chronic skin disorders. Properly administered local anesthesia usually makes this a painless procedure.

The biopsy site on the patient is numbed using a cream or spray before a local anesthetic is injected. Once the area is completely numb, the health professional cleans the biopsy site with an antiseptic wipe. The punch skin biopsy device is gently inserted into the skin, rotated, and a small circle of skin is carefully removed from the biopsy site. This biopsy sample will be sent to a laboratory for examination under a microscope and/or growth of cells from the underneath surface of the sample. The biopsy site usually bleeds during and immediately after the procedure, with blood obscuring the operative field. The area may be held closed by adhesive bandages or sutures, to stop further bleeding, since drying blood may adhere to the site and contribute to excessive scarring. The area may then be covered with a pressure dressing to stop any further bleeding.

A standard punch biopsy has several drawbacks. First, the metal cylindrical blade obscures the view of the lesion to be excised once the cutter is placed on the skin. It is important for the user of the biopsy punch to center the lesion within the punch area to make sure that the biopsy sample margins are evenly distributed around the lesion. This is very difficult to do when one cannot see the lesion once the punch is placed on the skin. Second, the retrieval of the biopsy is tedious. In addition to requiring a careful spearing of a very small biopsy sample with a needle or skin hook, the surgical field of the standard technique is usually filled with blood, which makes retrieval even more difficult. Spearing of the biopsy sample may also cause mechanical damage to the tissue, which can cause deleterious effects to diagnostic slide preparation. Third, portions of the biopsy sample may intentionally or accidentally remain in the cutter of the known biopsy punch. These portions of the biopsy sample must then be retrieved by hand, again with a delicate spearing by a needle or hook. This retrieval technique, as above, may result in damage to the biopsy sample.

Accordingly, it is desirable to provide a method and apparatus of a biopsy punch which: allows for precise positioning in the biopsy area, provides easy retrieval of the biopsy sample, avoids damage to the biopsy sample, may be used in a timely and efficient manner, and is economical to manufacture and use.

### SUMMARY OF THE INVENTION

In an embodiment of the present invention, a biopsy punch for removing a biopsy sample from a biopsy area of a patient is described. The biopsy punch includes a handle and a tubular blade. The handle is adapted for gripping by a user. The tubular blade extends around a longitudinal axis and has first and second longitudinally spaced blade ends separated by a blade body. The tubular blade is operatively coupled to the handle, has a cutting edge at the second blade end, has a plurality of openings located in the blade body and allowing the user to view the biopsy area for positioning the cutting edge in the biopsy area, and has at least one retainer prong extending toward the longitudinal axis and operative to engage the severed sample to assist in retaining the severed sample in the blade body. The tubular blade is adapted to sever at least a portion of the biopsy sample from the biopsy area and retain the severed sample in the blade body.

In an embodiment of the present invention, a method of removing a biopsy sample from a biopsy area of a patient is described. A biopsy punch including a handle adapted for gripping by a user, a tubular blade extending around a longitudinal axis and having first and second longitudinally spaced blade ends separated by a blade body is provided. The tubular blade is operatively coupled to the handle, has a cutting edge at the second end, and has a plurality of openings located in the blade body. The biopsy punch is positioned adjacent the biopsy area. The biopsy area is viewed through at least one of the plurality of openings. The biopsy sample is contacted with the cutting edge. The tubular blade is rotated in a first rotation direction—i.e., clockwise—about the longitudinal axis and moved longitudinally into the biopsy area. The biopsy sample is at least partially severed with the cutting edge. The biopsy sample is retained within the blade body. The biopsy sample is removed from the biopsy area.

In an embodiment of the present invention, a biopsy punch for removing a biopsy sample from a biopsy area of a patient is described. The biopsy punch includes a handle adapted for gripping by a user. A tubular blade extends around a longitudinal axis and has first and second longitudinally spaced blade ends separated by a blade body. The tubular blade is operatively coupled to the handle, has a cutting edge at the second blade end, has a plurality of openings located in the blade body, and has at least one retainer prong associated with an opening and operative to engage the severed sample to assist in retaining the severed sample in the blade body. The tubular blade is adapted to sever at least a portion of the biopsy sample from the biopsy area and retain the severed sample in the blade body.

### BRIEF DESCRIPTION OF THE DRAWINGS

For a better understanding of the invention, reference may be made to the accompanying drawings, in which:

FIG. 1 is a perspective view of one embodiment of the present invention;

FIG. 2 is a perspective view of the embodiment of FIG. 1;

FIG. 3 is a partial perspective view of the embodiment of FIG. 1;

FIG. 4A is a partial perspective view of the embodiment of FIG. 1 in a first mode;

FIG. 4B is a partial perspective view of the embodiment of FIG. 1 in a second mode;

FIG. 4C is a partial perspective view of the embodiment of FIG. 1 in a third mode;

FIG. 5A is a side view of the embodiment of FIG. 1 in the first mode;

FIG. 5B is a side view of the embodiment of FIG. 1 in the second mode;

FIG. 5C is a side view of the embodiment of FIG. 1 in the third mode; and

FIGS. 6A-6F illustrate the sequence of operation of the embodiment of FIG. 1.

### DESCRIPTION OF EMBODIMENTS

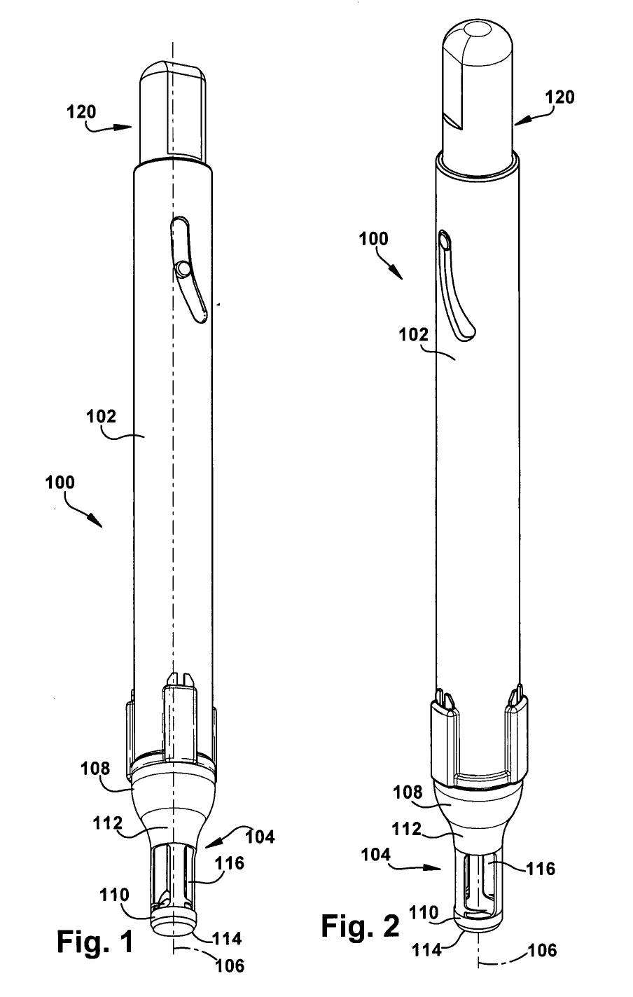

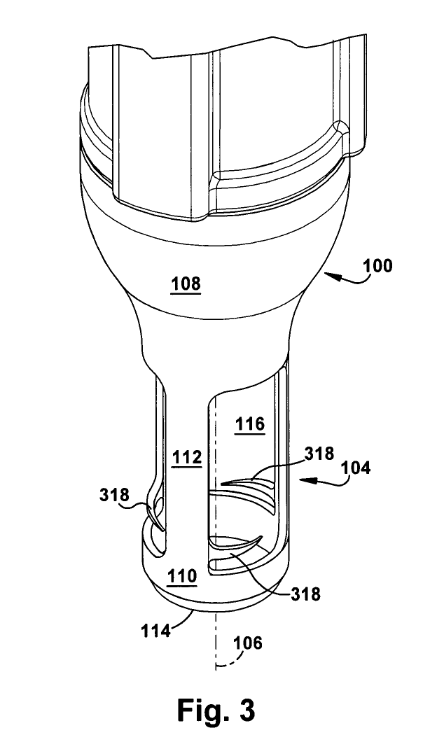

In accordance with the present invention, FIGS. 1 and 2 depict a biopsy punch 100. The biopsy punch 100 includes a handle 102 adapted for gripping by a user. The handle 102 may be designed with or without ergonomic considerations.

The biopsy punch 100 also includes a tubular blade 104. The tubular blade 104 extends around a longitudinal axis 106. The tubular blade has first and second longitudinally spaced blade ends 108 and 110, respectively, separated by a blade body 112. The tubular blade 104 is operatively coupled to the handle 102, optionally as a single piece or by the snap-fit connection with the first blade end 108 shown in the Figures. When the tubular blade 104 is provided as a separate piece from the handle 102, the tubular blade 104 could be disposable and the handle 102 could be adapted for re-use.

A cutting edge 114 is located at the second blade end 110. The cutting edge 114 could be oriented on an inward or outward side of the tubular blade 104. Particularly when the tubular blade 104 is intended for multiple uses, the cutting edge 114 may be designed and oriented for easy resharpening. The cutting edge 114 may be serrated or undulating as desired, rather than having the smooth, round profile shown in the Figures.

The second blade end 110 may include a band, ring, pitted area, grooves, or other structure (not shown) adapted to hold active/dispensable therapeutic agents which may be applied to a biopsy area on the patient during operation of the biopsy punch 100. Alternately or additionally, the second blade end 110 could be impregnated with therapeutic agents of a desired type. In either case, these agents could be hemostatic or antibacterial agents, tissue glue, scar reduction material, or any suitable agent or combination thereof. Since the second blade end 110 has direct contact with any severed blood vessels at the biopsy site, such agents may be readily applied to place the agents deep into the wound created by the biopsy punch 100 in the biopsy area. Such agents may make for a cleaner surgical field and faster healing, as well. For example, the second blade end 110 could hold chitosan in transferable powder form that can slough off in the wound, when the biopsy punch 100 cuts into the biopsy area. Chitosan acts as a hemostatic agent and has some tissue glue-like properties. Therefore, the wound will stop bleeding faster (less clotting factors) and the edges will approximate (glue together) more securely with only an adhesive bandage on the skin surface when chitosan is used, thus reducing or eliminating the need for sutures to close the wound.

At least one opening 116 is located in the blade body 112. The openings 116 allow a user to view the biopsy area, for positioning the cutting edge 114 as desired in the biopsy area. The openings 116 improve the accuracy of the biopsy and give the user a confirmation that the correct size biopsy punch 100 has been chosen for the desired biopsy sample size and any surrounding margins required. Multiple openings 116, when present, allow the biopsy area to be viewed from several angles simultaneously. If the user is unhappy with the visual margins around the biopsy sample, the openings 116 facilitate easy repositioning of the biopsy punch 100 within the biopsy area or determination that another biopsy punch 100 size should be chosen before a less than optimal biopsy sampling procedure is carried out.

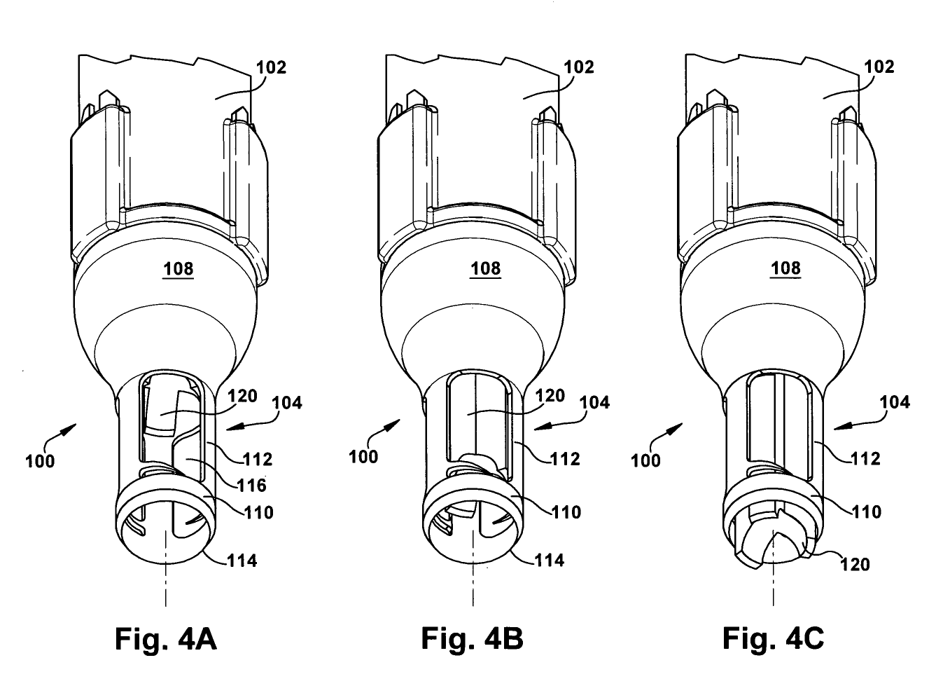

The tubular blade 104 is adapted to sever at least a portion of the biopsy sample (not shown) from the biopsy area and retain the severed sample in the blade body 112. At least one retainer prong 318 (shown in detail in FIG. 3) can engage the severed sample to assist in retaining the severed sample in the blade body 112. To do so, the retainer prongs hold onto the sample as the blade body is drawn upward from the biopsy area, thus allowing the user to manually sever the sample from the base layers of skin beneath the biopsy sample. The retainer prongs 318 may be deflected inward toward the longitudinal axis from the surface of the tubular blade 104. Each retainer prong 318 may be associated with an opening 116, as shown in the Figures, or may be located at a position on the tubular blade 104 spaced apart from the openings 116. Once completely severed from the patient, the biopsy sample will remain in the biopsy punch 100.

The biopsy punch 100 may include a sample ejector 120 adapted to selectively eject the biopsy sample being retained in the blade body 112. As shown in the Figures, the sample ejector 120 extends through the length of the handle 102 and tubular blade 104, much like the mechanism in a ballpoint pen. However, any suitable sample ejector 120 structure could be used with the biopsy punch 100 of the present invention.

Once removed from the patient, the biopsy sample is normally placed into a sample container with formalin solution for fixing. The sample ejector 120 is used to push the biopsy sample away from the retainer prongs 318, when present, and eject the biopsy sample from the second blade end 110 of the biopsy punch 100. The second blade end 110 may be placed directly above an open biopsy sample container and the sample ejector 130 actuated to drop the tissue sample into the solution without requiring further manipulation of the biopsy sample.

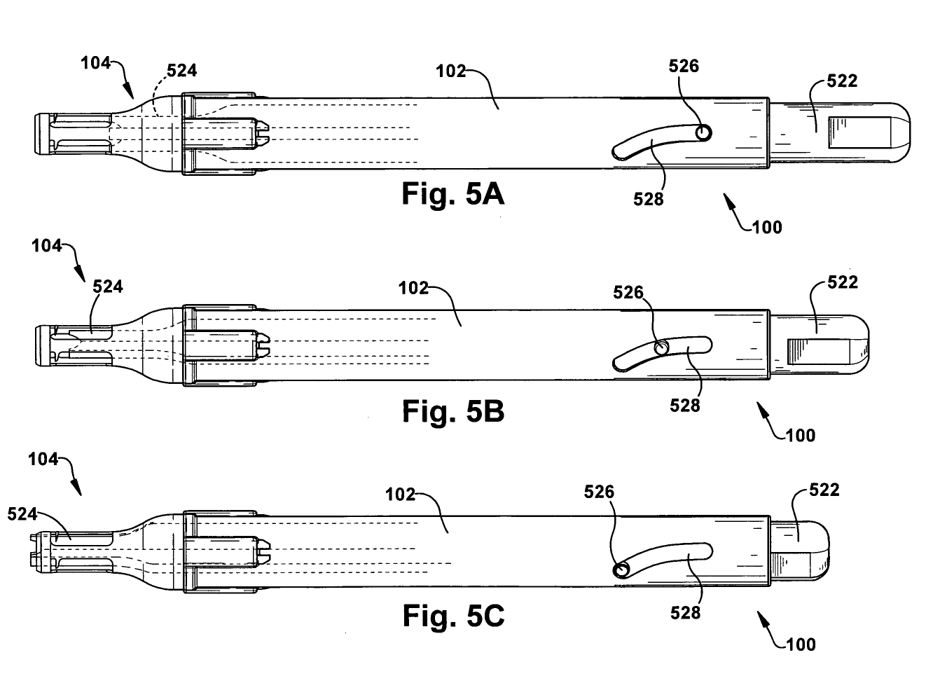

The positions of the sample ejector 120 are shown in greater detail in FIGS. 4A, 4B, and 4C. The sample ejector 120 is selectively moveable between a first position, shown in FIG. 4A, and a second position, shown in FIG. 4C, as well as a plurality of intermediate positions between the first and second positions, with a sample intermediate position shown in FIG. 4B. In the first position, the sample ejector 120 is fully retracted to allow retention of the severed sample in the blade body 112. In the second position, the sample ejector 120 is fully extended and the biopsy sample is pushed out of the blade body 112. As shown in FIG. 4C, the full extension of the sample ejector 120 in the second position optionally results in the sample ejector 120 protruding from the second blade end 110 and thus protecting the cutting edge 114 of the tubular blade 104 from inadvertent contact—in such case, the biopsy punch 100 may include a mechanism, such as a feature of the pin slot 528 engaging the ejector pin 526, to maintain the sample ejector 120 in the second position. Alternately, the sample ejector 120 may have a second position in which the biopsy sample is pushed out of the blade body 112 but the sample ejector 120 does not protrude from the second blade end 110.

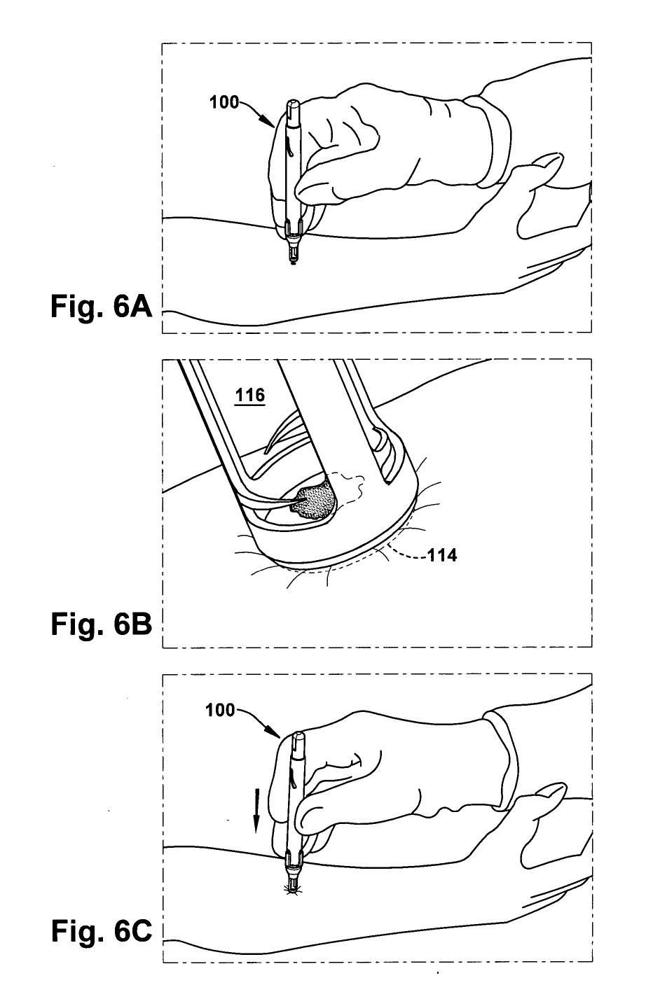

FIGS. 5A, 5B, and 5C illustrate the movement of the sample ejector 120 between the first, intermediate, and second positions, respectively. A plunger 522 is part of the sample ejector 120 and is operatively connected to an ejector tip 524 (shown in phantom line in FIG. 5A and in solid line in FIGS. 5B and 5C), which is the portion of the sample ejector 120 actually pushing the biopsy sample out of the tubular blade 104. The sample ejector 120 also includes an ejector pin 526 adapted to engage with a pin slot 528 in the handle 102 to indicate the position of the sample ejector 120. The pin slot 528 may be configured into a certain shape, such as the curve shown in the Figures, to direct movement of the sample ejector 120 through engagement with the ejector pin 526. The sample ejector 120 may be spring-biased into either the first or second position, in which case the engagement of the ejector pin 526 in the pin slot 528 may overcome the bias to hold the sample ejector 120 in a desired position.

The ejector pin 526 may be moved relative to the pin slot 528 through movement of the plunger 522, which also serves to move the ejector tip 524. Alternately, the sample ejector 522 need not include a plunger 522 if the ejector tip 524 is moved relative to the tubular blade 104 through user manipulation of the ejector pin 526 in the pin slot 528.

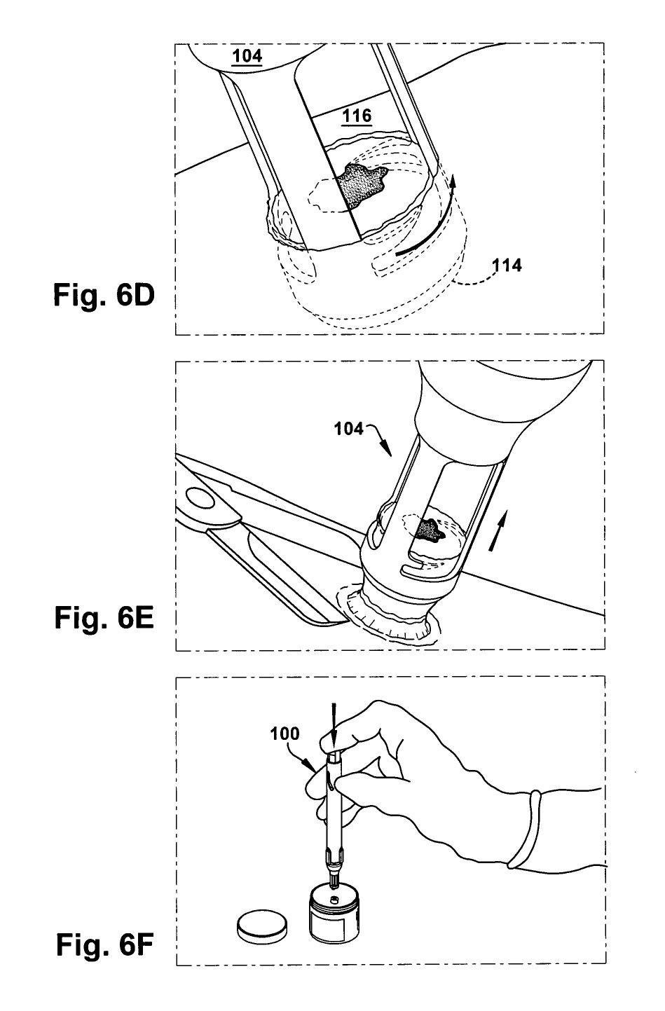

The operation of the biopsy punch 100 will now be described with reference to FIGS. 6A-6F. In FIG. 6A, the target lesion is identified and the correct size biopsy punch 100 is chosen. The biopsy punch 100 is placed gently on the biopsy area surrounding the desired biopsy sample (shown as a lesion in FIGS. 6A-6F). In FIG. 6B, the desired biopsy margins are verified by visual inspection through the openings 116. In FIG. 6C, the biopsy punch 100 is rotated in a first direction (here, clockwise) and downward into the biopsy area to cut the tissue to the desired depth. In FIG. 6D, the biopsy punch 100 is rotated in a second direction, opposite the first direction (here, counterclockwise), to engage the biopsy sample with the retainer prongs 318. In FIG. 6E, the biopsy punch 100 is gently lifted to retract the biopsy sample from the tissue bed of the biopsy area. The user may manually sever the biopsy sample at the desired depth (i.e., in the fat layer) if the biopsy sample is not completely severed as desired by the cutting edge 114. In FIG. 6F, the sample ejector 120 is operated to eject the biopsy sample into the tissue transportation container, optionally by the user's depressing the plunger 522. The ejector pin 526 may follow a path described by the pin slot 528, such as the arc or curve shown in the Figures, to rotate the sample ejector 120 as the biopsy sample is ejected. This rotation during ejection may help the sample ejector 120 to disengage the biopsy sample from the retainer prongs 318.

While aspects of the present invention have been particularly shown and described with reference to the preferred embodiment above, it will be understood by those of ordinary skill in the art that various additional embodiments may be contemplated without departing from the spirit and scope of the present invention. For example, the various elements of the biopsy punch 102 could be made of any suitable materials, including but not limited to plastics, metals, or any other materials. The retainer prongs 318 may be stamped/cut and bent inward from the body material of the tubular blade 104 or may be separately provided or attached. The sample ejector 120 could be fully retracted into the handle 102 in the first position or a portion of the sample ejector 120 could remain in the tubular blade 104 in the first position. The pin slot 528 may have any suitable shape to engage the ejector pin 526 and thereby rotate the sample ejector 120 in a desired manner. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of: allowing for precise positioning in the biopsy area, providing easy retrieval of the biopsy sample, avoiding damage to the biopsy sample, being usable in a timely and efficient manner, and being economical to manufacture and use. Such advantages are particularly worthy of incorporating into the design, manufacture, and operation of biopsy punches. In addition, the present invention may provide other advantages which have not yet been discovered.

Other aspects, objects, and advantages of the present invention can be obtained from a study of the drawings, the disclosure, and the appended claims.

Having described the invention, the following is claimed:

1. A biopsy punch for removing a biopsy sample from a biopsy area of a patient, the biopsy punch comprising:
a handle adapted for gripping by a user; and
a tubular blade extending around a longitudinal axis and having first and second longitudinally spaced blade ends separated by a blade body, the tubular blade being operatively coupled to the handle, having a cutting edge at the second blade end, having a plurality of openings located in the blade body and allowing the user to view the biopsy area for positioning the cutting edge in the biopsy area, and having at least one retainer prong extending toward the longitudinal axis and operative to engage the severed sample to assist in retaining the severed sample in the blade body;
the tubular blade being adapted to sever at least a portion of the biopsy sample from the biopsy area and retain the severed sample in the blade body.

2. The biopsy punch of claim 1, including a sample ejector, the sample ejector being selectively moveable between a first position, a second position, and a plurality of intermediate positions between the first and second positions, wherein the sample ejector is fully retracted to allow retention of the severed sample in the blade body in the first position, and the sample ejector is fully extended to eject the severed sample from the blade body in the second position.

3. The biopsy punch of claim 2, wherein the sample ejector protrudes from the second blade end when fully extended, to protect the cutting edge of the tubular blade.

4. The biopsy punch of claim 1, wherein the second blade end is adapted to deliver a therapeutic agent to the biopsy area.

5. The biopsy punch of claim 2, wherein at least one of the sample ejector and the second blade end is adapted to deliver a therapeutic agent to the biopsy area.

6. A method of removing a biopsy sample from a biopsy area of a patient, the method including the steps of:
providing a biopsy punch including a handle adapted for gripping by a user, a tubular blade extending around a longitudinal axis and having first and second longitudinally spaced blade ends separated by a blade body, the tubular blade being operatively coupled to the handle, having a cutting edge at the second blade end, and having a plurality of openings located in the blade body;
positioning the biopsy punch adjacent the biopsy area;
viewing the biopsy area through at least one of the plurality of openings;
contacting the biopsy sample with the cutting edge;
rotating the tubular blade in a first rotation direction about the longitudinal axis;
moving the tubular blade longitudinally into the biopsy area;
at least partially severing the biopsy sample with the cutting edge;
retaining the biopsy sample within the blade body; and
removing the biopsy sample from the biopsy area.

7. A biopsy punch for removing a biopsy sample from a biopsy area of a patient, the biopsy punch comprising:
a handle adapted for gripping by a user;
a tubular blade extending around a longitudinal axis and having first and second longitudinally spaced blade ends separated by a blade body, the tubular blade being operatively coupled to the handle and having a cutting edge at the second blade end;
the second blade end being adapted to deliver a pharmaceutical agent to the biopsy area.

8. A biopsy punch for removing a biopsy sample from a biopsy area of a patient, the biopsy punch comprising:
a handle adapted for gripping by a user; and
a tubular blade extending around a longitudinal axis and having first and second longitudinally spaced blade ends separated by a blade body, the tubular blade being operatively coupled to the handle, having a cutting edge at the second blade end, having a plurality of openings located in the blade body, and having at least one retainer prong associated with an opening and operative to engage the severed sample to assist in retaining the severed sample in the blade body;
the tubular blade being adapted to sever at least a portion of the biopsy sample from the biopsy area and retain the severed sample in the blade body.

9. The biopsy punch of claim 8, including a sample ejector, the sample ejector being selectively moveable between a first position, a second position, and a plurality of intermediate positions between the first and second positions, wherein the sample ejector is fully retracted to allow retention of the severed sample in the blade body in the first position, and the sample ejector is fully extended to eject the severed sample from the blade body in the second position.

10. The biopsy punch of claim 9, wherein the sample ejector protrudes from the second blade end when fully extended, to protect the cutting edge of the tubular blade.

11. The biopsy punch of claim 8, wherein the second blade end is adapted to deliver a therapeutic agent to the biopsy area.

12. The biopsy punch of claim 9, wherein at least one of the sample ejector and the second blade end is adapted to deliver a therapeutic agent to the biopsy area.

13. The biopsy punch of claim 8, wherein at least one opening allows the user to view the biopsy area for positioning the cutting edge in the biopsy area.

14. The biopsy punch of claim 8, wherein at least one retainer prong extends toward the longitudinal axis.
